(19) United States
(12) Patent Application Publication
(10) Pub. No.: US 2013/0131567 A1
(43) Pub. Date: May 23, 2013

(54) BACK BRACE SYSTEM

(71) Applicants: Patrick Scott Hinshon, Maplewood, MN (US); Thomas Kramer, Blaine, MN (US)

(72) Inventors: Patrick Scott Hinshon, Maplewood, MN (US); Thomas Kramer, Blaine, MN (US)

(21) Appl. No.: 13/472,882

(22) Filed: May 16, 2012

Related U.S. Application Data
(60) Provisional application No. 61/488,438, filed on May 20, 2011.
(60) Provisional application No. 61/622,204, filed on Apr. 10, 2012.

(51) Int. Cl. A61F 5/02

(52) U.S. Cl. CPC A61F 5/028
USPC 602/19

(57) ABSTRACT

A back brace for support of a patient's back having a pair of belts that together distract the spine.

### CROSS REFERENCE

The present case claims the benefit of and incorporates by reference in their entirety several U.S. Provisional Applications viz: 61/488438, filed 2011 May 16 entitled “Back Brace and Method”; 61/411669, filed 2012 Mar. 16 entitled “Enhanced Back Brace”; and 61/622204, filed 2012 Apr. 10 entitled “Adjustable Back Brace”.

### FIELD

This invention relates to orthotic back braces for treating a patient's back.

### BACKGROUND

There are many back braces available however there is a continuing need to improve these devices to meet patient needs for an adjustable, comfortable device that permits patient motion to promote healing while supporting regions of the patients back and distracting the patient's spine.

### SUMMARY

The primary brace system comprises two overlapping, connected but independent belts or bands. In use these are wrapped around the patient's waist. Once donned the two belts may be tensioned separately. The first inner belt or band defines a waist groove on the patient when it is tightened. Typically the inner belt has low area so that it “digs in” to the patient's waist. Once positioned the inner belt is relatively stable on the patients body, and does not move from its location as the patient moves about. The second outer belt typically overlies most of the inner belt and when cinched it compresses adipose tissue. The relatively larger area of the second outer belt then can compress the displaced tissue and distracts or stretch the spine. The two belts cooperating together serve to apply the right amount of force on the patient's abdomen at the right areas to effectively relieves back pain from surgery or the like.

In an alternative embodiment the inner belt or band is used for a short time during fitting of the brace system to form thermal panels or thermally re-formable panels to “fit” lateral panels to the patient defining a waist groove.

Several optional features may be added to the brace system. For example the small of the back can be fitted with a fixed formed back panel or an adjustable back panel. The adjustable version of this feature may supply a structure that flexes inward toward the back “filling” that space between the belt and that portion of the patient anatomy. In either embodiment the back panel improves transmission of force to the body from the belts and helps to anchor the two belts to a relatively stable position on the patient.

Another optional feature is the inclusion of a belly panel located between the patient's abdomen and either the first inner belt or the second outer belt. The preferred version of the belly panel has automatically adjustable area so that it automatically adjust to the patients changes in posture, increasing in area as the patient stands up and reduces in area as the patient returns to a seated position. The optional features may be used in any combination or permutation with the remainder of the belt and brace system.

For Example in a usual configuration there will be an inner band, an outer belt, a lumbar support structure and an abdominal pad. Each of these items may be used in isolation in an alternative back brace but they work well together to provide an efficient solution to patient indications.

### DRAWINGS

Throughout the figures identical reference numeral indicate identical structure wherein:

FIG. 1 is a schematic view of a patient;

FIG. 2 is a schematic view of a patient with an inner belt or band fitted;

FIG. 3 is a schematic view of the patient with both belts and an abdominal pad fitted;

FIG. 4 is a schematic view of an implementation of the brace system as viewed from the inside;

FIG. 5 is a schematic view of an implementation of the brace system as view from the outside;

FIG. 6 is a schematic view of a patient donning a brace system;

FIG. 7 is a schematic view of a patient wearing a brace system;

FIG. 8 is a schematic view of a patient wearing a brace system;

FIG. 9 is an exploded schematic diagram of a brace system;

FIG. 10 is a perspective view of a lumbar panel;

FIG. 11 is a perspective view of a lumbar panel;

FIG. 12 is a perspective view of a brace system in isolation;

FIG. 13 is a perspective view of a brace system in isolation;

FIG. 14 is a schematic view of an alternate embodiment of the back brace system being fitted;

FIG. 15 is a schematic view of an alternate embodiment of the brace system being fitted;

FIG. 16 is a perspective and exploded view of an alternate brace system;

FIG. 17 is a perspective view of the alternate embodiment of the brace system assembled;

FIG. 18 is a perspective view of an alternate embodiment of the brace system on a patient;

FIG. 19 is a perspective view of an abdominal panel; and,

FIG. 20 is a perspective view of an abdominal panel.

### DETAILED DESCRIPTION

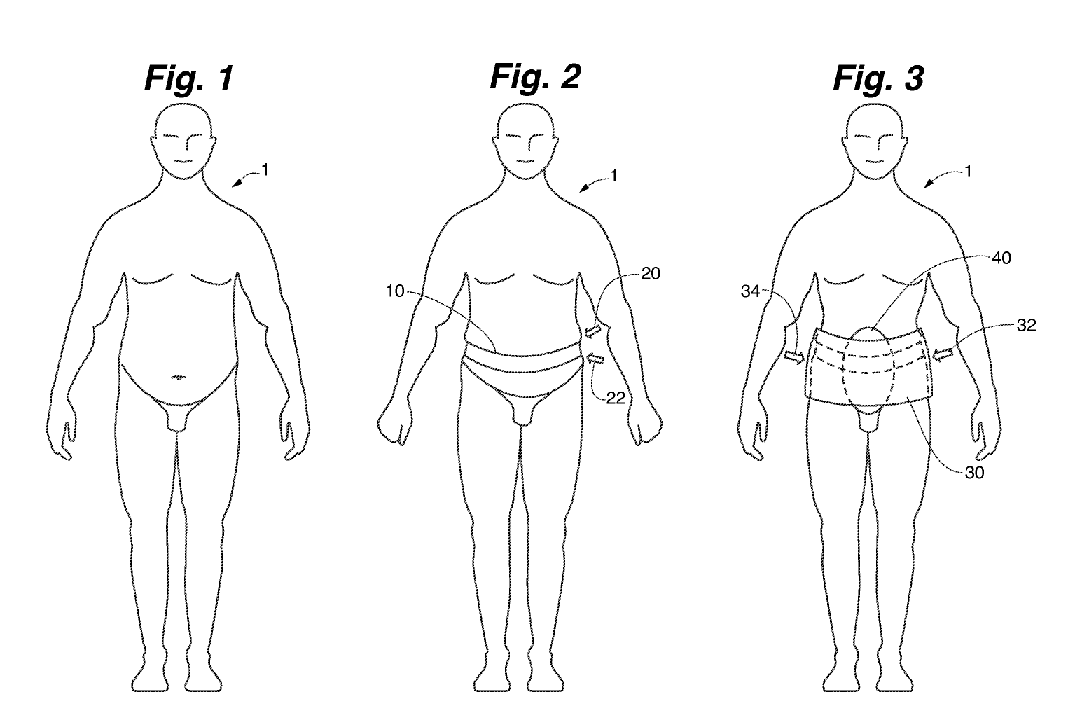

FIG. 1, FIG. 2, and FIG. 3 illustrate the operation of one version of the system in a schematic form. When the patient 1 of FIG. 1 dons the inner band 10 and tightens it he displaces tissue as indicated in FIG. 2 by the arrows at reference numeral 20 and reference numeral 22. With the outer belt 30 donned over the inner band 10 as seen in FIG. 3 the patient 1 tightens the complimentary outer belt thereby compressing the displaced tissue of FIG. 2 as indicated in FIG. 3 by arrows 32 and 34. This compressive force on the abdomen distracts the spine that provides comfort and relieves the back pain. For a patient with a pendulous belly an optional abdominal pad 40 may be placed against the belly as well to improve containment, control and distraction.

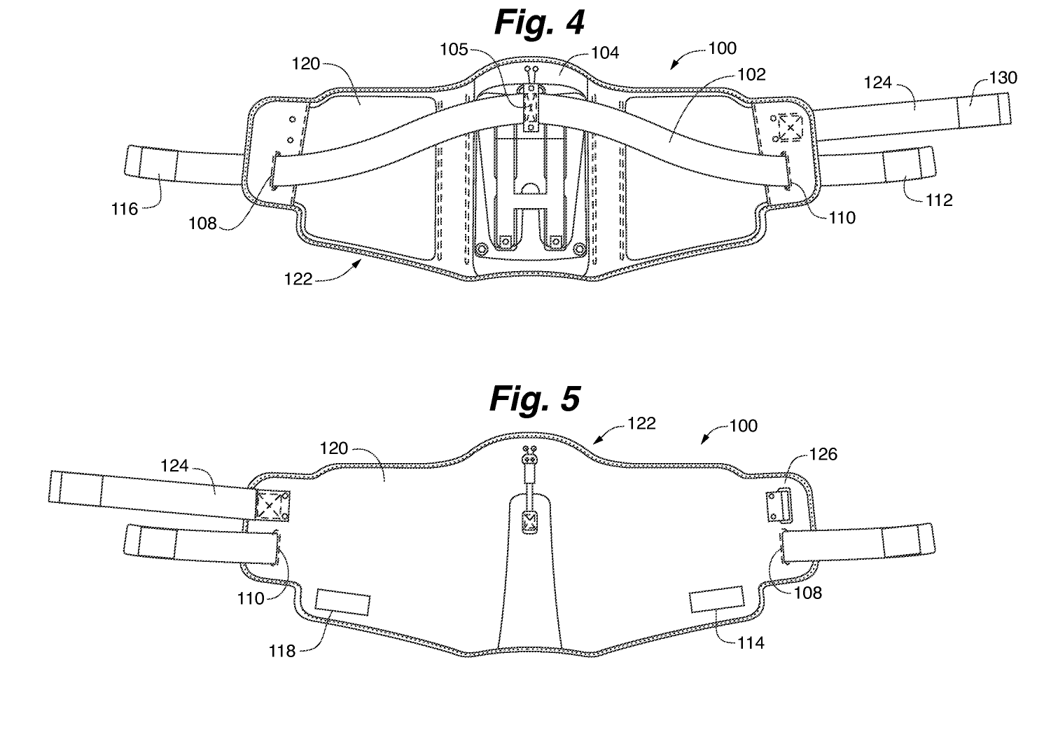

FIG. 4 is a schematic view of an implementation of the brace system 100 laid out flat in the plane of the paper and as seen from the inside, while FIG. 5 is a schematic view of the brace system 100 as seen from the outside or back while laid out in a plane. In this embodiment the inner belt band 102 is a length webbing attached to and anchored to the lumbar panel 104 by a stitching to a strap 105. The inner band or belt is then freely threaded through the outer belt 120 at left aperture 108 and right aperture 110. The inner band 102 once tensioned is fixed to anchor zones on the outer belt. For example a hook and loop clasp pad 112 may mate with pad 114 when the brace is donned by crossing in front of the patient. In a similar fashion pad 116 may mate with pad 118 by crossing in the front of the patient. In prototype form the pads are made from the familiar Velcro style hook and loop material.

The outer belt 120 is a laminated or composite structure encasing the lumbar panel 104 and terminating in a tensioning member 124. The tensioning member in this embodiment is length of webbing that may be passed though a loop anchor 126 doubled over and fixed with a hook and loop pad 130 to connect to itself (124). In essence the length of the tension member passed through the loop anchor 126 sets the size or circumference of the belt.

FIG. 6 is a schematic figure showing the patient 1 donning the brace system 100. The left side and right side are wrapped around the torso as indicated by arrows 132 and 134.

FIG. 7 is a schematic view of the brace system 100 on a patient 1 as seen from the front. In this figure the tensioning member is closed on itself as indicated at reference numeral 138 and the inner belt 102 has been crossed over the torso and anchored to the anchor points indicated at reference numeral 140 and complimentary reference numeral 142. This view indicates that the tension in the two belts may be adjusted separately. In use the patient may adjust the two belts in any order to customize the fit.

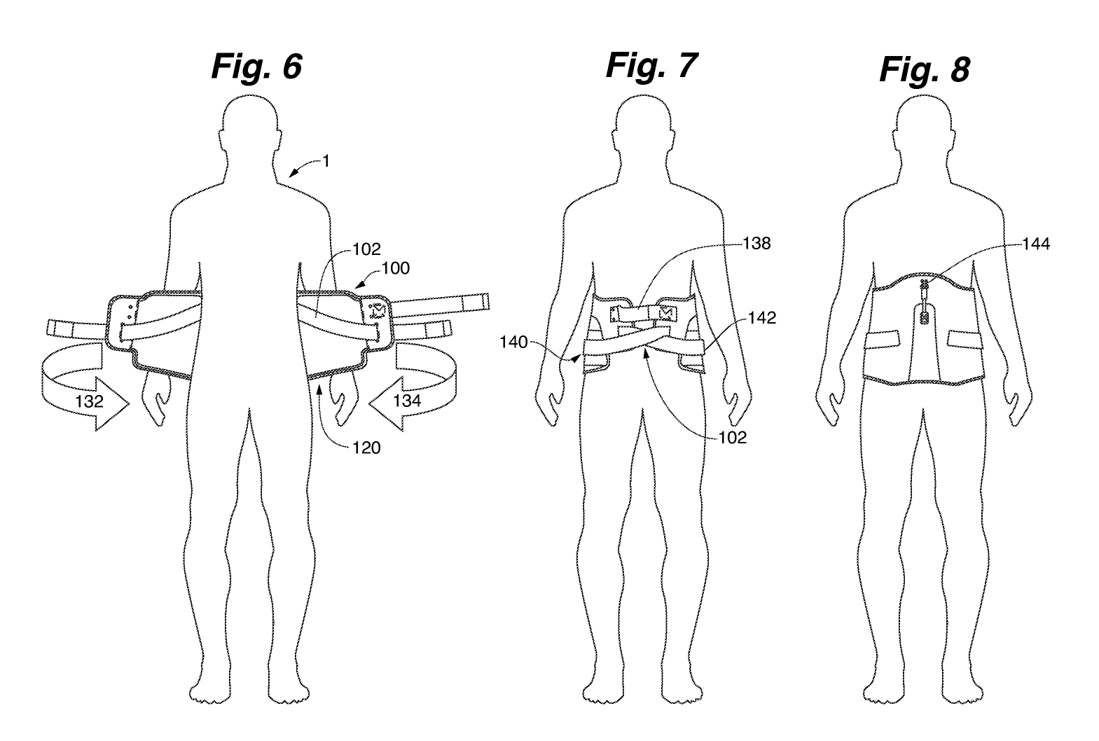

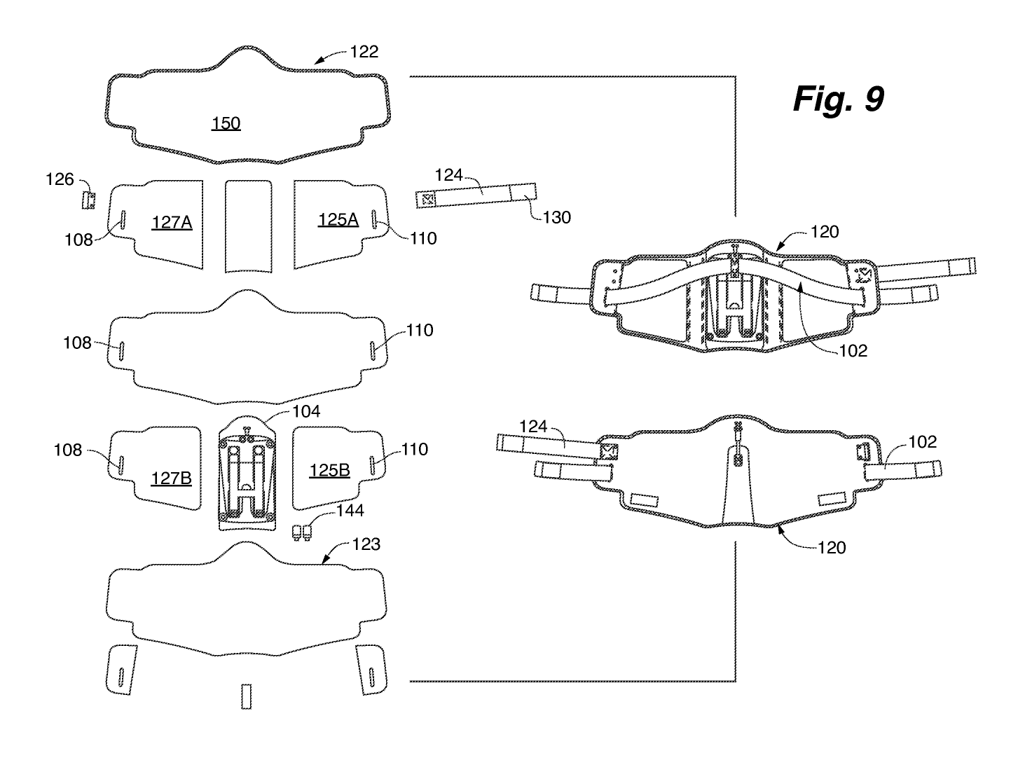

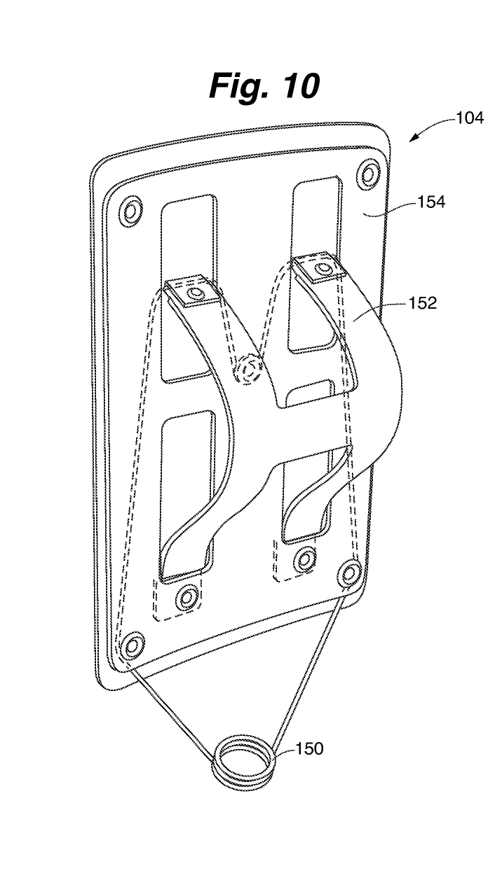

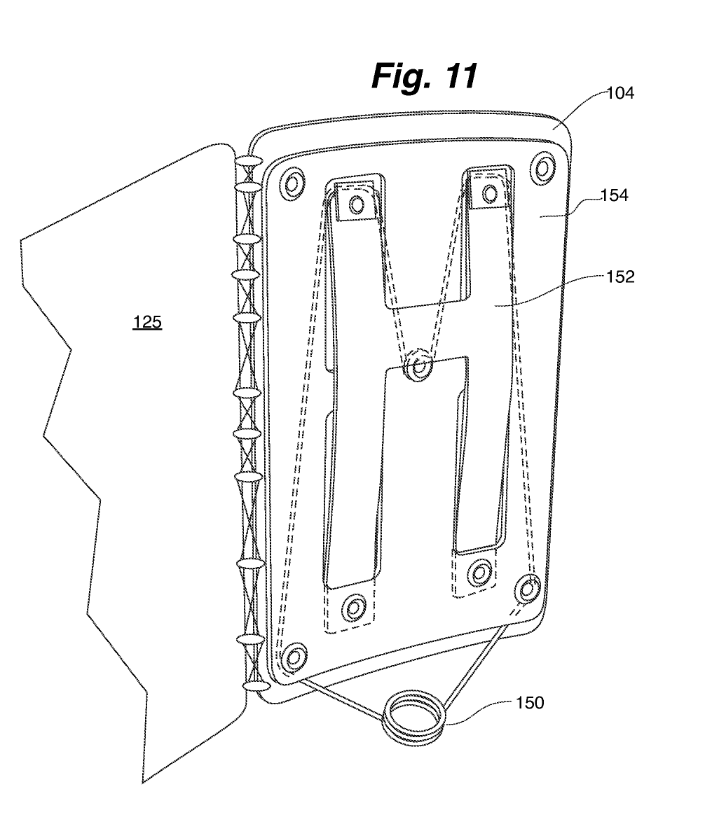

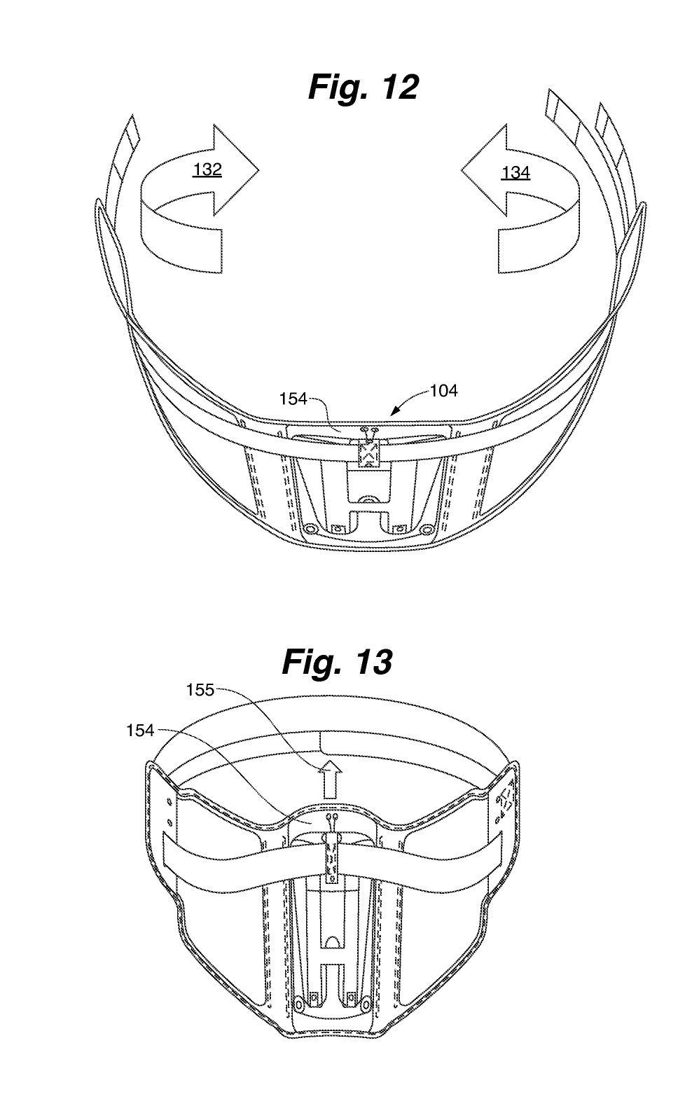

FIG. 8 is a schematic view of the brace system of FIG. 7 as seen from the rear. A tensioning tab 144 is seen in this figure and it is used to activate the lumber panel 104 and further described in connection with FIG. 10 and FIG. 11.

FIG. 9 is an exploded drawing of the parts that make up one representative version of one embodiment of the invention depicted in FIG. 4 and FIG. 5. The outer belt 120 is made of several independent piece parts assembled as seen in the figure. For example the outer cover 122 and inner cover 123 are sewn to inner lateral panel. These elements are attached to the lumbar panel 104 125A and 125 B as well as lateral panel 127A and 127B.

FIG. 10 is a perspective view of a lumbar panel 104. In this version of the lumbar support an H-shaped batten 152 may flex out of the plane of the support 154 toward the small of the back. This may be accomplished by a cable and pulley system as depicted in the figure. A reel type winder 150 may take up the cable slack moving the batten 152. As an alternative the cables may be exteriorized and a Velcro pull tab 144 may be used to set the flex of the batten as seen in other figures.

FIG. 11 is a perspective view of a lumbar panel 104, in a relaxed state and attached to a lateral side panel 125 via a lacing or sewn construction. The remaining structures are omitted for clarity.

FIG. 12 is a perspective view of the lumbar panel 104 during the donning process the support panel 154 is sufficiently flexible that it can flex to accommodate the shape of the of the lumbar panel 104. Which is seen in FIG. 13 and depicted in the figure by arrow 155 showing curvature of the support panel 154 as the two belts are tensioned as indicated by closure arrows 132 and closure arrow 134.

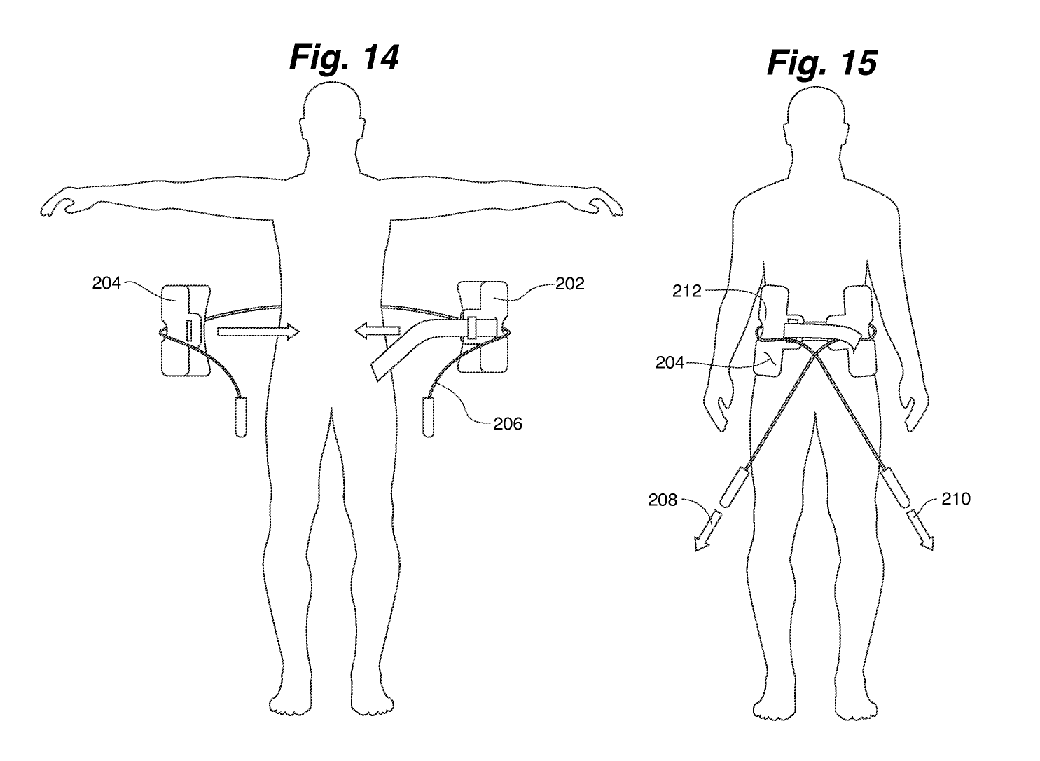

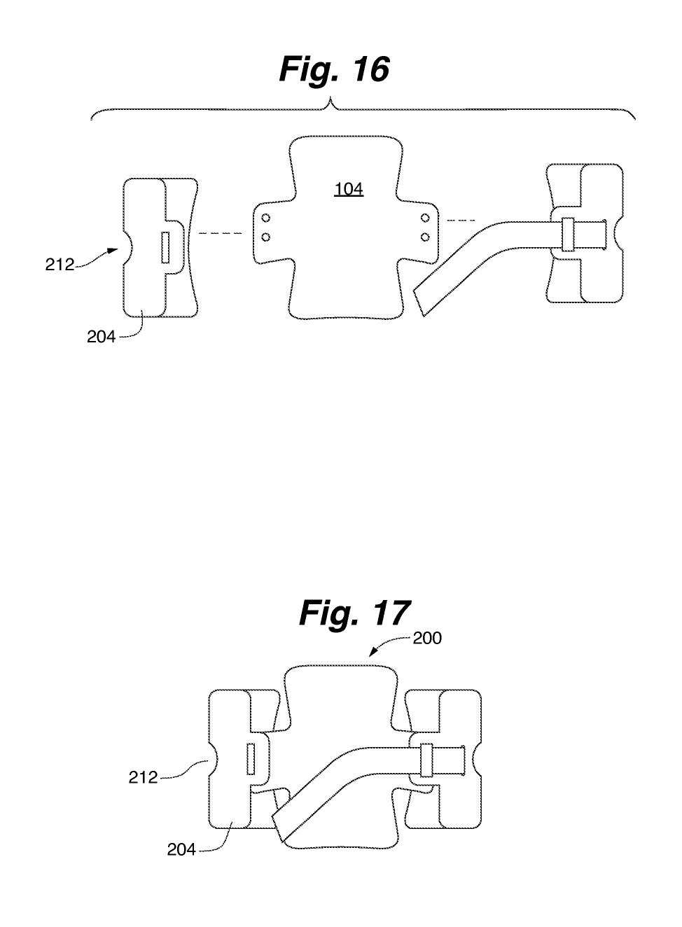

FIG. 14 is a schematic view of an alternate embodiment of the back brace system in the process of fitment. In this example two lateral panels 202 and 204 are formed of a thermally formable or re-formable material and they are positioned around the patients waist as indicated by motion arrows in the figure. A “jump rope” cable 206 is looped around the waist and tensioned as indicated by tension arrows 208 and 210 as seen in FIG. 15 to draw the panels into close conformity to the patient and make a waist groove seen by indentation 212 in panel 204 in FIG. 15.

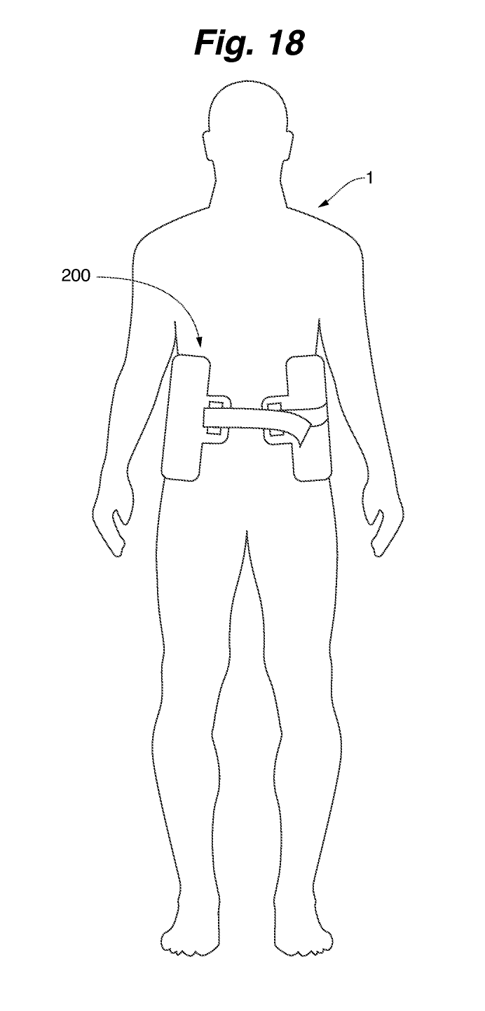

FIG. 16 is a perspective and exploded view of an alternate brace system where the panels heated and fitted to the patient in the process described in connection with FIG. 14 and FIG. 15 are assembled or connected to a lumbar panel 104 in FIG. 16 to form the completed brace 200 seen in isolation in FIG. 17.

FIG. 18 is a perspective view of the alternate embodiment of the brace 200 as described in FIG. 14 through FIG. 17 in place on a patient.

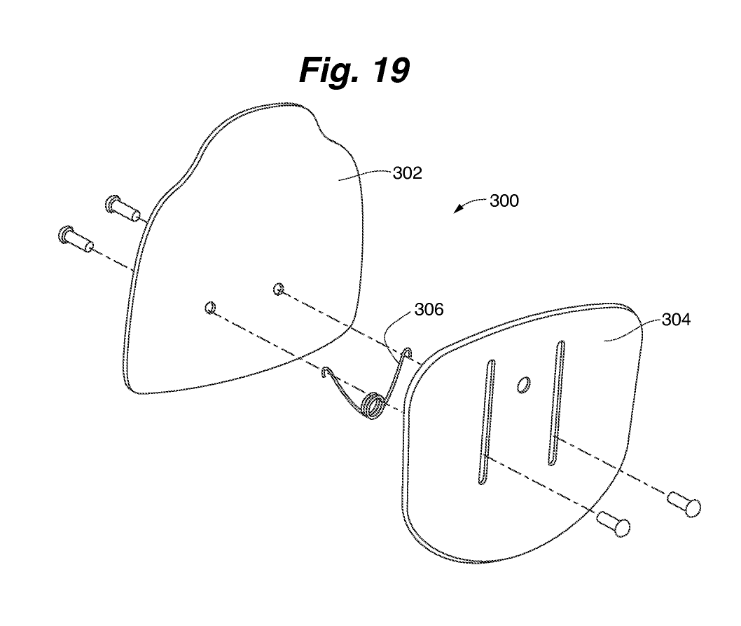

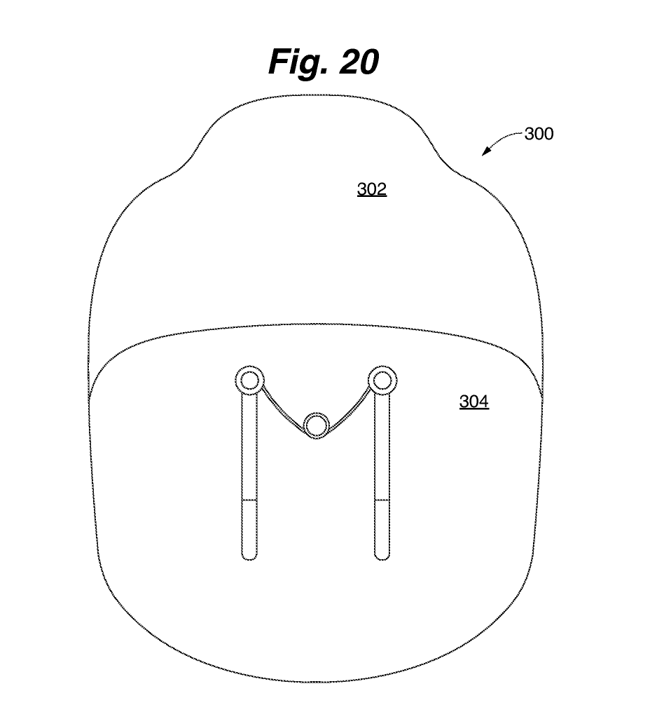

FIG. 19 and FIG. 20 are perspective views of an abdominal panel assembly 300 in the assembled configuration in FIG. 20 and in an exploded view in FIG. 19. As seen in the figures the abdominal panel assembly 300 is preferably made from two pieces shown as upper panel 302 and lower panel 304, that engage each other and are free to move with the patient so that the projected area can have a first value with the patient seated and a second larger value while the patient stands. This motion allows the distraction effects to be more uniform in the standing and seated states.

What is claimed:

1. An orthotic back brace device comprising:
a first belt having a first area, and having a first clasp to secure the belt at a first circumference;
a second belt having a second area, said second area greater than the first area, and having a closure to secure the belt at a second circumference.

2. The back brace of claim 1 wherein said second circumference is larger than said first circumference.

3. The back brace of claim 1 wherein said first belt includes a back panel, a pair of lateral side panels, said panels connected to each other forming an outer belt overlying said first belt.

4. The back brace of claim 2 wherein said first and second belt are connected together to allow independent movement with respect to each other.

5. The back brace of claim 1 wherein said first closure includes a mechanical advantage to permit the user to apply force to said first belt.
